(19) United States
(12) Patent Application Publication
(10) Pub. No.: US 2024/0057610 A1
(43) Pub. Date: Feb. 22, 2024

(54) USE OF EXTRACELLULAR MEMBRANE VESICLES FOR ANTI-BIOFILM PURPOSES

(71) Applicant: Université de Bourgogne, DIJON CEDEX (FR)

(72) Inventors: Aurélie RIEU, Dijon (FR); David DA SILVA BARREIRA, Dijon (FR); Jean GUZZO, Fontaine les Dijon (FR)

(21) Appl. No.: 18/258,199

(22) PCT Filed: Dec. 17, 2021
(86) PCT No.: PCT/FR2021/052374

§ 371 Date: Jun. 16, 2023

(30) Foreign Application Priority Data
Dec. 18, 2020 (FR) 2013717

(51) Int. Cl. A01N 63/20; C12N 1/20

(52) U.S. Cl. CPC A01N 63/20; C12N 1/205; C12R 2001/225

(57) ABSTRACT

The present invention relates to a use of extracellular membrane vesicles of at least one probiotic for preventing or reducing the formation of a biofilm on the surface of a material. The present invention also relates to a method for treating a surface of a material in order to prevent or reduce the formation of a biofilm on the surface, the method comprising a step of bringing extracellular membrane vesicles derived from at least one probiotic into contact with the surface. The present invention further relates to a material comprising extracellular membrane vesicles of at least one probiotic on its surface, or into which extracellular membrane vesicles of at least one probiotic are incorporated.

### CROSS-REFERENCE TO RELATED APPLICATIONS

The present application is a filing under 35 U.S.C. 371 as the National Stage of International Application No. PCT/FR2021/052374, filed Dec. 17, 2021, entitled “USE OF EXTRACELLULAR MEMBRANE VESICLES FOR ANTI-BIOFILM PURPOSES,” which claims priority to French Application No. 2013717 filed with the Intellectual Property Office of France on Dec. 18, 2020, both of which are incorporated herein by reference in their entirety for all purposes.

### TECHNICAL FIELD

The present invention relates to the use of biological tools for preventing or reducing the formation of a biofilm on the surface of a material.

The present invention finds applications in many fields of industry, such as for example in the food processing industry, the pipe or surface treatment industry, or in the medical sector.

In the following description, references enclosed in brackets ([ ]) refer to the list of references presented at the end of the text.

### STATE OF THE ART

Biofilms are in the form of a viscous film consisting of microorganisms, often bacteria, yeasts, fungi or algae.

The formation of a biofilm is a dynamic and non-fixed process which takes place in several steps detailed below:
reversible adhesion: planktonic bacteria (isolated bacteria, in a suspension) arrive near the substrate by gravity, by diffusion or owing to dynamic flows and adhere reversibly by means of physical/chemical phenomena such as electrostatic forces, Van der Waals forces, and hydrophobic interactions;
irreversible adhesion: the production of extracellular polymers and the placement of bonds between the bacterial appendages (pili, flagella, adhesion proteins) and the surface allow the irreversible adhesion of the bacteria to the surface. Removing irreversibly adhered cells is difficult;
microcolony formation: the bacteria aggregate and multiply to form microcolonies. The bacterial cells within the microcolonies are bound in a matrix of extracellular polymeric substances (EPS). The EPSs are produced in response to adhesion and environmental stimuli such as pressure, pH, temperature or nutrient depletion in the medium.
maturation: microcolonies develop and the biofilm is structured in a three-dimensional manner Several conditions affect the structure of this mature biofilm such as the surface, the composition of the microbial community, the availability of nutrients, and the hydrodynamic conditions.

Biofilms are formed on the surfaces of industrial equipment and colonize all industrial surfaces, such as pipes, or membrane filters. Several studies have demonstrated that, during certain operations, in particular in plate exchangers, biofilms can be formed if the manufacturers are not particularly vigilant. They are thus commonly responsible for severe contaminations of finished products and numerous instances of food poisoning. Biofilms are for example a major concern in the dairy processing industry. Bacterial biofilms may also develop on implants or during chronic infections; they act as reservoirs of pathogens and may cause nosocomial infections.

The ability of bacteria obtained from biofilms to adhere is very large (often greater than that of planktonic cells) either on natural or artificial surfaces. Bacteria within the biofilms are resistant to any sort of stress, which makes them difficult to eliminate. Biofilms are very good at withstanding chemical stresses (antibiotics, for example) and mechanical stresses (fluids, for example). Thus, biofilms withstand most conventional cleaning methods, and have a tendency to develop more in water or in aqueous media. However, many factors promote the formation of biofilms such as temperature or access and the nature of the available metabolic resources.

Current strategies to limit the formation of a biofilm are diverse (Rendueles O & Ghigo J M ([1]); Venkatesan N, Perumal G & Doble M ([2]); Rao P K & Sreenivasa M Y ([3])). Some of these strategies are proactive and aim to prevent the adhesion and formation of the biofilm One of these strategies is the use of synthetic signaling molecules, which interfere with the cell communication system that is essential for the biofilm to form. This last application is still at the exploratory stage.

The use of nanotechnologies for the prevention of biofilms is currently an important strategy. In particular, it involves the incorporation of antibacterial agents into inert media. The release of nanoparticles is nevertheless to be taken into account depending on the applications.

Other strategies are curative and aim to eradicate biofilms. These are bactericidal methods, or methods for dispersing and disaggregating a biofilm that has already formed. Bactericidal methods can be divided into physical, chemical, and biological approaches.

Physical and mechanical treatments, such as ionizing radiation, UV radiation and ultrasound, have been experimented with in the past. Their efficacy is partial, but it is possible to combine these methods to potentiate the anti-biofilm effect. In the case of ultrasound treatments, many unwanted effects have been reported relating the quality of the food, its physical composition, and its flavor.

The use of disinfectants, in particular for fresh products, is frequent.

However, such solutions are much more active on planktonic cells. The bacteria present in biofilms have increased resistance to disinfectants. Indeed, the organization within the matrix network that the biofilm represents affords the bacteria effective protection against chemical agents. On the one hand, the biofilm constitutes a permeability barrier making it possible to reduce the exposure of bacteria to chemical agents, and on the other hand the bacteria present in the biofilms have reduced metabolic activities, thus making it possible to further reduce the action of the chemical agents. In the food industries, surfaces are cleaned with chlorinated derivatives, hydrogen peroxide, iodine, isothiazolinones, ozone, peracetic acid, acid compounds, aldehyde-based biocides, phenols, biguanides, surfactants, halogens and quaternary ammoniums. In general, these agents do not completely eliminate the biofilm, are not eco-friendly, and cause surface corrosion in many cases. The effect of essential oils on the destruction of biofilms is also being studied.

The use of bacteriophages is currently being developed for their bactericidal property.

Recently, the incorporation of biosurfactants in liposomes has been proposed with anti-biofilm activity. In this case, it involves reconstituted artificial vesicles. Effects were demonstrated against a S. aureus biofilm, for potential applications relating to skin diseases.

Moreover, enzymes are used to disperse the biofilm. This is mainly hydrolases (a-amylases, proteases, ribonucleases for example), oxidoreductases (such as glucose-oxidases or haloperoxidases), transferases (such as transaminase) or lyases (such as alginate lyase). Since these enzymes do not kill bacteria, they are generally combined with bactericidal methods.

There is therefore a real need for tools that overcome the defects, drawbacks and obstacles of the prior art, in particular a tool to prevent or reduce the formation of a biofilm on the surface of a material.

### DESCRIPTION OF THE INVENTION

Through major research, the Applicant has succeeded in demonstrating that the use of membrane vesicles, which are naturally produced by probiotics, prevents biofilm formation by pathogenic or unwanted microorganisms on biotic and abiotic surfaces.

Very advantageously, the vesicles are derived from probiotics, which provides an advantage for their use, in particular for health. This origin guarantees a safe, natural, eco-friendly, non-polluting product with a broad spectrum of action, which can even constitute a protective film, with preventive action.

Advantageously, several effects can be envisaged, including the anti-biofilm and immunomodulator effect, depending on the probiotic strain used to isolate the vesicles.

Thus, a first subject of the invention relates to the use of extracellular membrane vesicles of at least one probiotic for preventing or reducing the formation of a biofilm on the surface of a material.

For the purposes of the present invention, “extracellular membrane vesicles” means any vesicle of a lipid nature, released spontaneously or in induced manner (by culture conditions or by chemical treatments) in the medium by the probiotic, and containing at least one active ingredient belonging to this productive bacterium. Advantageously, it is conceivable to produce vesicles loaded with active ingredients which may be lipids, proteins, nucleic acids or exopolysaccharides.

For the purposes of the present invention, the term “probiotic” means any living microorganism which, when ingested in sufficient quantity, has a beneficial effect on the health of the host. It may in particular be bacteria or probiotic yeasts, in particular a bacterium such as a lactobacillus, a bifidobacterium, an enterococcus, a propionibacterium, a streptococcus and a bacterium of the genus Bacillus, or a yeast such as Saccharomyces cerevisiae and Saccharomyces boulardii or a mixture thereof. The probiotic bacteria can be selected from: L. acidophilus, L. crispatus, L. gasseri, L. delbrueckii, L. salivarius, L. casei, L. paracasei, L. plantarum, L. rhaninosus, L. reuteri, L. brevis, L. buchneri, L. fermentuni, B. adolescentis, B. angulation, B. bifidum, B. breve, B. catenulatuni, B. infantis, B. lactis, B. longum, B. pseudocatenulatuni, S. therrnophilus, or a mixture thereof, preferably the probiotic bacteria are L. casei, L. paracasei and L. plantarum or a mixture thereof. The probiotic yeasts suitable for the present invention may be selected from: Saccharomyces cerevisiae and Saccharomyces boulardii or a mixture thereof.

According to the invention, combinations of different types of vesicles, originating for example from different types of probiotic bacteria, can be carried out. For example, it is possible to use vesicles from one or more different bacterial species, the number of different species not being limited. It may for example be a mixture of L. casei and L. paracasei. Optionally, the vesicles can be used in combination with at least one antimicrobial with curative or preventive properties, that the person skilled in the art may choose from known antimicrobials depending on the targeted application.

For the purposes of the present invention, the term “biofilm” means a multicellular community of microorganisms adhering together and to a surface, and secreting an adhesive and protective EPS matrix.

According to the invention, the biofilm may be a bacterial biofilm, a yeast biofilm or a mixed biofilm. For the purposes of the present invention, the term “bacterial biofilm” means a biofilm whose multicellular community of microorganisms consists essentially of bacteria. For the purposes of the present invention, the term “yeast biofilm” means a biofilm whose multicellular community of microorganisms consists essentially of yeasts. For example, the bacterial biofilm may be formed by at least one bacterial species selected from the family of enterobacteria, in particular Salmonella enterica Enteritidis, Hafnia alvei and/or Citrobacter freundii, the genus Staphylococcus, in particular Staphylococcus aureus or Staphylococcus epidermidis, the genus Bacillus, in particular Bacillus cereus or Bacillus subtilis, the genus Pseudomonas, in particular Pseudomonas aeruginosa and the genus Enterococcus, in particular Enterococcus faecalis. For example, the yeast biofilm can be formed by the yeast species Candida albicans. Within the meaning of the present invention, “mixed biofilm” means a biofilm composed of a community of different types of microorganisms, which may in particular comprise bacteria, yeasts and/or phages. For example, the bacterial biofilm may comprise at least one bacteria selected from the family of enterobacteria, in particular Salmonella enterica Enteritidis, Hafnia alvei and/or Citrobacter freundii, the genus Staphylococcus, in particular Staphylococcus aureus or Staphylococcus epidermidis, the genus Bacillus, in particular Bacillus cereus or Bacillus subtilis, the genus Pseudomonas, in particular Pseudomonas aeruginosa and the genus Enterococcus, in particular Enterococcus faecalis, and from the yeast species Candida albicans.

For the purposes of the present invention, the term “preventing the formation of a biofilm” means the action of preventing, on a biofilm-free surface, the formation of biofilm. In particular, the vesicles prevent the adhesion of bacteria on the treated surface. The preventive effect of the extracellular membrane vesicles may take place for a period of time that can reach several weeks to several months after the treatment of the surface, in particular if the surface is conditioned with the vesicles and the material is stabilized, by drying for example.

“Reducing the formation of a biofilm”, within the meaning of the present invention, means the action of partly preventing, on a biofilm-free surface, the formation of biofilm. The reduction may be reducing at least 20% of the formation of a biofilm, relative to an identical surface preserved under the same conditions, in the absence of treatment. The biofilm formation-reducing effect of the extracellular membrane vesicles may take place over a period of time that can reach several weeks to several months after the treatment of the surface, in particular if the surface is conditioned with the vesicles and the material is stabilized, by drying for example.

Advantageously, the anti-biofilm effect of the extracellular vesicles does not, or does not necessarily, come with an antimicrobial effect. Indeed, antimicrobial activity is to be distinguished from anti-biofilm activity. Advantageously, the extracellular vesicles prevent bacterial adhesion to the treated surface, for reasons independent of any antimicrobial effects. This feature is even more advantageous when in certain cases it is desirable to have an anti-biofilm effect without having an antimicrobial effect.

The extracellular membrane vesicles may be produced according to any method known to the person skilled in the art. The production method may for example comprise the following steps:
(a) culturing at least one probiotic under conditions suitable for the production of extracellular membrane vesicles,
(b) separating the at least one probiotic and extracellular membrane vesicles produced in step (a) and,
(c) purification and concentration of the extracellular membrane vesicles.

The culture step (a) can be carried out under standard conditions known to a person skilled in the art, depending on the nature of the probiotic. For example, in the case of lactobacilli, the culture can be carried out in the MRS medium, at 37° C., for 24 h.

The separation step (b) can also be carried out under standard conditions known to a person skilled in the art, depending on the nature of the probiotic. For example, it may involve a filtration step. For example, in the case of lactobacilli, centrifugation can be carried out at 4000 g for 20 minutes and the filtration can be carried out with a filter having a pore size of about 0.22 pm.

The purification and concentration step (c) can be carried out under standard conditions known to a person skilled in the art, depending on the nature of the probiotic.

The purification and concentration step may comprise at least one technique known to a person skilled in the art, such as differential centrifugation, illustrated by Zaborowska et al. ([4]), density gradient, illustrated by Kim et al. ([5]) or Dean et al. ([6]), exclusion chromatography, illustrated by Kuhn et al. ([7]), ultrafiltration, illustrated by Mata Forsberg et al. ([8]), Dominguez Rubio et al. ([9]), Choi et al., 2020 ([10]) or Kim et al. ([5]), immunocapture (IC), illustrated by Wubbolt et al. ([11]), such as for example the IC on a column, by magnetic beads coupled to antibodies or any other surface coupled to a specific antibody, or precipitation, as illustrated by Bäuerl et al. ([12]) this list is not limiting. In the case of lactobacilli, ultrafiltration can be an exclusion filtration at approximately 100 kDa. Advantageously, step (c) can make it possible to obtain a solution of vesicles with a concentration of about 10¹¹ particles/mL, this number being given by way of indication and able to vary according to the conditions for implementing the different steps of the protocol c) and according to the vesicles producing the vesicles.

The stabilization and preservation of the extracellular membrane vesicles can be carried out under standard conditions known to a person skilled in the art. For example, the stabilization and preservation step may comprise a drying and/or freezing step.

The material on which the extracellular membrane vesicles are used may be any material on which a biofilm is likely to form. It may in particular be a material selected from metals, metal alloys, polymers, glass, ceramic, and food.

The extracellular membrane vesicles may be incorporated into products making it possible to treat these materials. It may for example be a spray, or a covering product like a paint, lacquer or varnish.

In the context of the treatment of a surface according to the invention, the use is understood to mean an ex-vivo, non-therapeutic use.

Another object of the invention relates to a method for treating a surface of a material for preventing or reducing the formation of a biofilm on said surface, said method comprising a step of contacting extracellular membrane vesicles from at least one probiotic with said surface.

For the purposes of the present invention, “treatment” means the application of a layer of extracellular membrane vesicles on the surface of the material. The application in particular takes place under normal conditions of use of the material, for example at room temperature and at atmospheric pressure. The amount of vesicles applied to the surface can be determined by a person skilled in the art, depending on the material and type of vesicle.

Another object of the invention relates to a material comprising, on its surface, extracellular membrane vesicles of at least one probiotic.

According to the invention, the vesicles at least partially cover the surface of the material to be protected, and preferably they do so completely. They can thus form a layer, with a thickness that can be between 10 and 500 nm. The thickness will be determined by a person skilled in the art as functions of applications.

The material may be a packaging material, in particular food packaging, a water pipe, a heat exchanger, a pipeline, or a catheter. The material may also be a material used in the medical sector, because in addition to the anti-biofilm activity, the vesicles may provide anti-inflammatory activity (Mata Forsberg et al. ([8]), Kim et al. ([13]), Nahui Palomino et al. ([14]), Yamasaki-Yashiki et al. ([12]), (Bauerl et al. ([12]), Choi et al. ([10]), Kuhn et al. ([7]). Thus, the vesicles may be incorporated into dressings, creams or cover certain medical devices.

Other advantages may be seen by the person skilled in the art by reading the following examples, shown by the appended figures provided by way of illustration.

### BRIEF DESCRIPTION OF THE FIGURES

FIG. 1 represents the diagram illustrating the steps of the protocol produced in examples 1 and 2.

FIG. 2 shows a portion of the spectrum of action for the anti-biofilm effect of the extracellular vesicles of L. casei BL23 (dotted) and L. paracasei ATCC334 (close dotted lines). Quantification (as a percentage relative to NT) of the formation of a biofilm of the pathogens S. aureus (FIG. 2A), S. epidermidis (FIG. 2A), H. alvei (FIG. 2B), S. enterica (FIG. 2B), E. faecalis (FIG. 2C), P. aeruginosa (FIG. 2C) and B. subtilis (FIG. 2D) is determined either without adding extracellular vesicles (close hatched lines), or with the addition of 0.04 μg/μL of vesicles. Control is carried out with the vesicles purified from the MRS culture medium (hatched lines). The formation of the biofilm by the pathogens after 24 h at 37° C. is quantified by crystal violet staining. The condition without adding extracellular vesicles (close hatched lines) corresponds to the condition NT (not treated). FIG. 3 shows an anti-biofilm effect of the vesicles of L. casei BL23 and L. paracasei ATCC334 against S. aureus, S. epidermidis, H. alvei, S. enterica, E. faecalis, P. aeruginosa and B. subtilis.

FIG. 3A and FIG. 3B shows: A) the quantification (as a percentage relative to NT) the formation of a biofilm of S. enterica, either without the addition of extracellular vesicles (close hatched lines), or with the addition of 0.04 μg/μL extracellular vesicles of L. casei BL23 (dots), or with the addition of 0.04 μg/μL of vesicles purified from the MRS culture medium (hatched lines). The formation of the biofilm by S. enterica after 24 h at 37° C. is quantified by crystal violet staining, the condition without adding extracellular vesicles (close hatched lines) corresponds to the condition NT (not treated); B) the growth curve of S. enterica followed by reading the OD at 600 nm for 24 h at 37° C. for the following conditions: without the addition of extracellular vesicles of the L. casei BL23 (round), or with the addition of 0.04 μg/μL extracellular vesicles L. casei BL23 (triangle), or with the addition of 0.04 μg/μL of vesicles purified from the MRS culture medium (square). FIG. 3 shows that the anti-biofilm effect of the vesicles of L. casei BL23 against S. enterica does not come with an antimicrobial effect.

FIG. 4 represents the quantification (as a percentage relative to NT) of the formation of the biofilm by S. enterica at 0 h, 4 h, 8 h and 15 h at 37° C. by crystal violet staining S. enterica either was not treated with vesicles (close hatched lines), or was treated by 0.04 μg/μL of extracellular vesicles L. casei BL23 (dots), or was treated by 0.04 μg/μL of vesicles purified from the MRS culture medium (control, hatched lines). The condition without adding extracellular vesicles (close hatched lines) corresponds to the condition NT (not treated). FIG. 4 shows that the anti-biofilm effect of the vesicles of L. casei BL23 against S. enterica relates to early steps of biofilm formation.

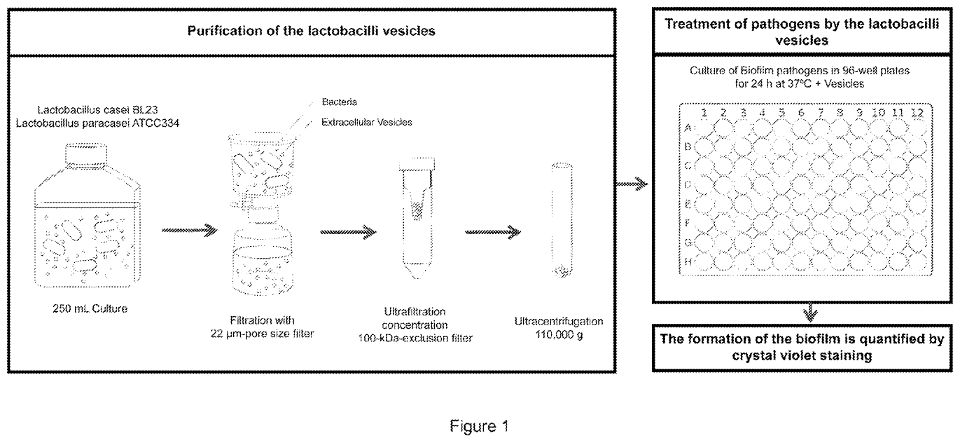

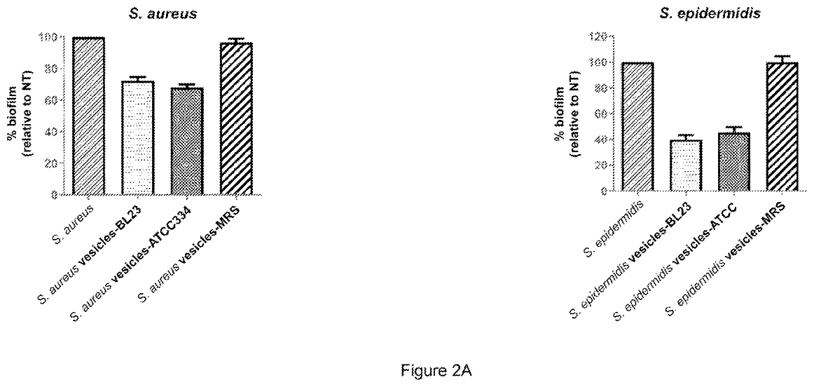

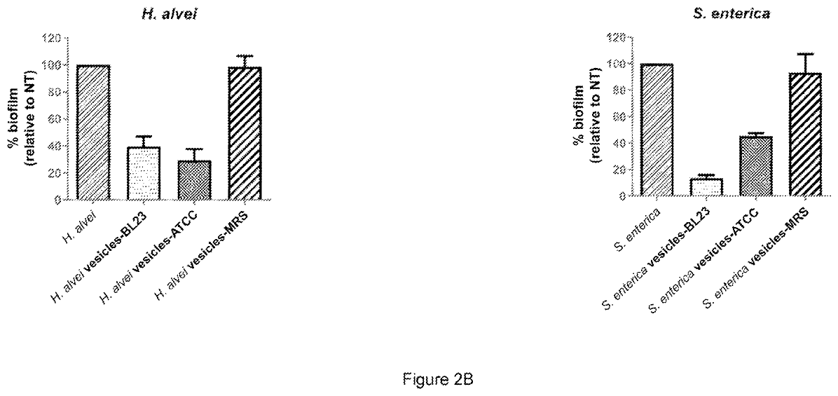

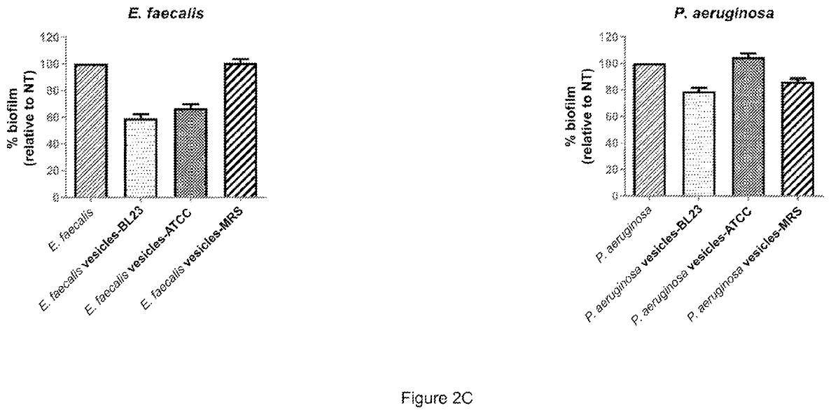

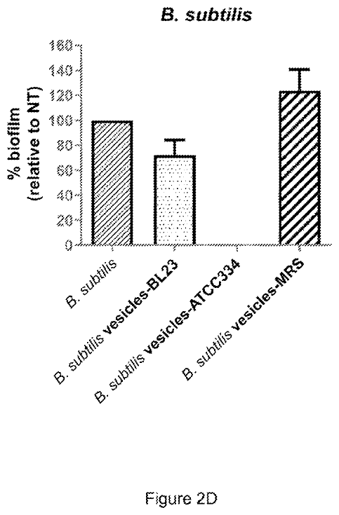

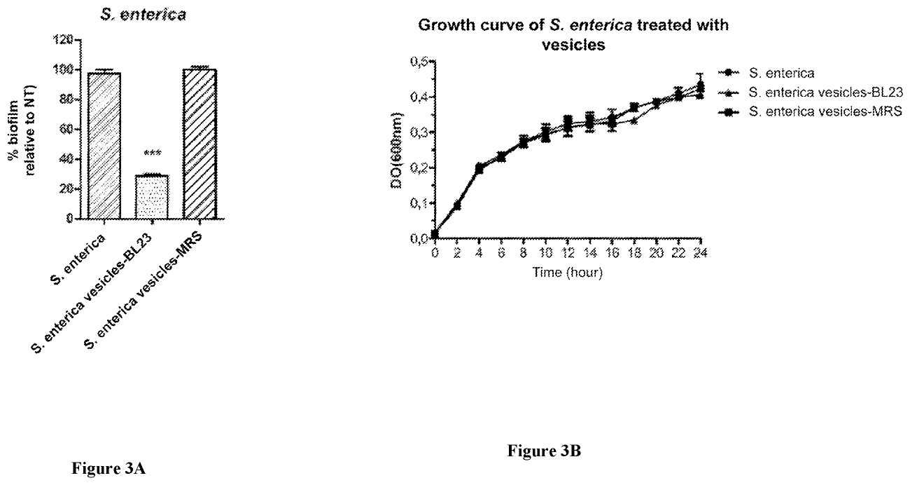

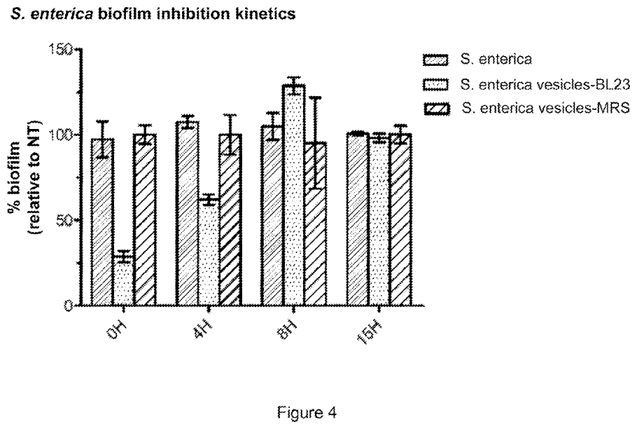

### EXAMPLES

### Example 1: Isolation of the Membrane Vesicles

Material:
MRS (Man, Rogosa, Sharpe) culture medium
0.22 μm PES (Polyethersulfone) filters
10 mL syringes
Pyrex 250 mL (sterile) vials
Centrifuge tube, 15 mL and 50 mL (sterile)
Ultrafiltration system (100-kDa-exclusion filter):
Amicon® Ultra (ref. UFC510008
Centricon Plug 70 (ref. UFC710008)
Heraeus Multifuge X3R centrifuge
Rotor swing-out BIOLiner adapter (11646190)
50 mL centrifuge tube adapter
250 mL vial adapter
OPTIMA L series 90K (NS COL 96J32) ultracentrifuge
Beckman SW41 Ti Swinging-Bucket Rotor (ref. 331362)
Thin-wall polypropylene tube 13.2 mL, 14×89 mm (ref. 33137)
Phosphate Buffered Saline (sterile)

Protocol

Step 1: Culturing of the Lactobacilli

A protocol for culturing the lactobacilli with standard parameters is proposed below.
Inoculate 15 mL of MRS with approximately 50 μL of a lactobacillus retained in 20% glycerol at 80° C. (cryotube outlet)
Incubate at 37° C., 24 h
Inoculate 15 mL of MRS with the cryotube outlet. Dilute to achieve an optical density at 600 nm of 0.05 (OD600 nm=0.05) (Pre-culture)
Incubate at 37° C., 24 h
Inoculate 250 mL of MRS at OD600 nm=0.05 with the preculture (Working culture)
Incubate at 37° C. for 24 h

Step 2: Concentration and Isolation of Vesicles by Filtration and Ultrafiltration

The biological material is kept at 4° C. under aseptic conditions during step 2
Centrifuge 250 mL to 4000 g for 20 min in order to precipitate the cells
Collect the clarified supernatant in a 250 mL sterile bottle
Filter the clarified supernatant through a 0.22 lam filter
Concentrate the supernatant using an ultrafiltration system (100-kDa-exclusion filter) (ref. UFC710008)
Concentrate 250 mL until a volume of 10-15 mL of liquid is obtained
Filter through a 0.22 lam filter (in order to remove the aggregates)
Ultracentrifuge the concentrated supernatant at 110,000 g for 2 h at 4° C.
Eliminate the supernatant and resuspend the pellet with 500 μL of PBS at 4° C. (fraction of concentrated vesicles)
Perform a protein assay (Bradford assay) of the concentrated vesicular fraction thus obtained
Keep the vesicular fraction cold.

### Example 2: Antibiotic Activity of Vesicular Faction of Lactobacilli

Material
Culture medium TSB
96-well flat bottom culture plate with 1 GREINER 2515432 lid
Pro-Lab Diagnostic™ crystal violet solution (ref. 12926287)

Protocol

Step 1: Formation of a Biofilm on Polystyrene Microplates and Treatment with the Fraction of Concentrated Vesicles
Inoculate 15 mL of TSB with approximately 50 μL of a bacteria retained in 20% glycerol at 80° C. (cryotube outlet)
Incubate at 37° C., 24 h (±stirring, ±aerobic depending on the bacterium studied)
Inoculate 15 mL of TSB with the cryotube outlet. Dilute to achieve an optical density at 600 nm of 0.05 (0D600 nm=0.05) (Pre-culture)
Incubate at 37° C., 24 h (±stirring, ±aerobic depending on the bacterium studied)
Inoculate 20 mL of TSB at OD600 nm=0.05 with the preculture (Working culture)
Distribute the working culture into the wells of a polystyrene plate and add the vesicular fraction to a final concentration of 0.04 μg/μL per 100 μL of final volume.
Incubate at 37° C., 24 h (±stirring, ±aerobic depending on the bacterium studied)

Step 2: Quantification of the Formation of the Biofilm by Staining with the Violet
Remove the suspended bacteria in each of the wells of the polystyrene plate
Wash each well of the polystyrene plate twice with 200 μL of distilled water
Add 150 μL of crystal violet at 0.5% (dilution in distilled water) in each well
Incubate 1 h without stirring
Wash each well of the polystyrene plate twice with 200 μL of distilled water
Add 150 μL of 95% ethanol (dilution in distilled water) in each well
Measure absorbance at 595 nm

### LIST OF REFERENCES

1. Rendueles O & Ghigo J M, Multi-species biofilms: how to avoid unfriendly neighbors FEMS Microbiol Rev 2012; 36: 972-989. DOI: 10.1111/j.1574-6976.2012.00328.x
2. Venkatesan N, Perumal G & Doble M, Bacterial resistance in biofilm-associated Bacteria Future Microbiol 2015; 10(11):1743-50. DOI: 10.2217/fmb.15.69
3. Rao P K & Sreenivasa M Y, Probiotic Lactobacillus Strains and Their Antimicrobial Peptides to Counteract Biofilm-Associated Infections-A Promising Biological Approach S M J Bioinform Proteomics. 2016; 1 (2): 1009.
4. Zaborowska, M., Taulé Flores, C., Vazirisani, F., Shah, F. A., Thomsen, P., Trobos, M., 2020. Extracellular Vesicles Influence the Growth and Adhesion of Staphylococcus epidermidis Under Antimicrobial Selective Pressure. Front. Microbiol. 11, 1132.
5. Kim, H., Kim, M., Myoung, K., Kim, W., Ko, J., Kim, K. P., Cho, E.-G., 2020. Comparative Lipidomic Analysis of Extracellular Vesicles Derived from Lactobacillus plantarum APsulloc 331261 Living in Green Tea Leaves Using Liquid Chromatography-Mass Spectrometry. Int. J. Mol. Sci. 21, 8076.
6. Dean, S. N., Rimmer, M. A., Turner, K. B., Phillips, D. A., Caruana, J. C., Hervey, W. J., Leary, D. H., Walper, S. A., 2020. Lactobacillus acidophilus Membrane Vesicles as a Vehicle of Bacteriocin Delivery. Front. Microbiol. 11, 710.
7. Kuhn, T., Koch, M., Fuhrmann, G., 2020. Probiomimetics—Novel Lactobacillus-Mimicking Microparticles Show Anti-Inflammatory and Barrier-Protecting Effects in Gastrointestinal Models. Small 16, 2003158.
8. Mata Forsberg, M., Bjorkander, S., Pang, Y., Lundqvist, L., Ndi, M., Ott, M., Escriba, I. B., Jaeger, M.-C., Roos, S., Sverremark-Ekstrom, E., 2019. Extracellular Membrane Vesicles from Lactobacilli Dampen IFN-γ Responses in a Monocyte-Dependent Manner. Sci. Rep. 9, 17109.
9. Dominguez Rubio, A. P., Martinez, J. H., Martinez Casillas, D. C., Coluccio Leskow, F., Piuri, M., Perez, O. E., 2017. Lactobacillus casei BL23 Produces Microvesicles Carrying Proteins That Have Been Associated with Its Probiotic Effect. Front. Microbiol. 8, 1783.
10. Choi, J. H., Moon, C. M., Shin, T.-S., Kim, E. K., McDowell, A., Jo, M.-K., Joo, Y. H., Kim, S.-E., Jung, H.-K., Shim, K.-N., Jung, S.-A., Kim, Y.-K., 2020. Lactobacillus paracasei-derived extracellular vesicles attenuate the intestinal inflammatory response by augmenting the endoplasmic reticulum stress pathway. Exp. Mol. Med. 52, 423-437.
11. Wubbolts, R., Leckie, R. S., Veenhuizen, P. T. M., Schwarzmann, G., Möbius, W., Hoernschemeyer, J., Slot, J.-W., Geuze, H. J., Stoorvogel, W., 2003. Proteomic and Biochemical Analyses of Human B Cell-derived Exosomes: POTENTIAL IMPLICATIONS FOR THEIR FUNCTION AND MULTIVESICULAR BODY FORMATION. J. Biol. Chem. 278, 10963-10972.
12. Bäuerl, C., Coll-Marqués, J. M., Tarazona-González, C., Pérez-Martinez, G., 2020. Lactobacillus casei extracellular vesicles stimulate EGFR pathway likely due to the presence of proteins P40 and P75 bound to their surface. Sci. Rep. 10, 19237.
13. Kim, W., Lee, E. J., Bae, 1.-H., Myoung, K., Kim, S. T., Park, P. J., Lee, K.-H., Pham, A. V. Q., Ko, J., Oh, S. H., Cho, E.-G., 2020. Lactobacillus plantarum-derived extracellular vesicles induce anti-inflammatory M2 macrophage polarization in vitro. J. Extracell. Vesicles 9, 1793514.
14. Nahui Palomino, R. A., Vanpouille, C., Laghi, L., Parolin, C., Melikov, K., Backlund, P., Vitali, B., Margolis, L., 2019. Extracellular vesicles from symbiotic vaginal lactobacilli inhibit HIV-1 infection of human tissues. Nat. Commun. 10, 5656.
15. Yamasaki-Yashiki, S., Miyoshi, Y., Nakayama, T., Kunisawa, J., Katakura, Y., 2019. IgA-enhancing effects of membrane vesicles derived from Lactobacillus sakei subsp. sakei NBRC15893. Biosci. Microbiota Food Health 38, 23-29.

1. A use of extracellular membrane vesicles of at least one probiotic for preventing or reducing the formation of a biofilm on the surface of a material.

2. The use according to claim 1, wherein the at least one probiotic is selected from a probiotic bacterium such as a lactobacillus, a bifidobacterium, an enterococcus, a propionibacterium, a streptococcus and a bacterium of the genus Bacillus, or a yeast such as Saccharomyces cerevisiae and Saccharomyces boulardi or a mixture thereof.

3. The use according to claim 1, wherein the biofilm is a bacterial biofilm, a yeast biofilm or a mixed biofilm.

4. The use according to claim 3, wherein:
said bacterial biofilm comprises at least one bacterial species selected from the family of enterobacteria, in particular Salmonella enterica Enteritidis, Hafnia alvei and/or Citrobacter freundii, the genus Staphylococcus, in particular Staphylococcus aureus or Staphylococcus epidermidis, the genus Bacillus, in particular Bacillus cereus or Bacillus subtilis, the genus Pseudomonas, in particular Pseudomonas aeruginosa and the genus Enterococcus, in particular Enterococcus faecalis
said yeast biofilm comprises the yeast species Candida Albicans
said mixed biofilm comprises a mixture of at least one bacteria selected from the family of enterobacteria, in particular Salmonella enterica Enteritidis, Hafnia alvei and/or Citrobacter freundii, the genus Staphylococcus, in particular Staphylococcus aureus or Staphylococcus epidermidis, the genus Bacillus, in particular Bacillus cereus or Bacillus subtilis, the genus Pseudomonas, in particular Pseudomonas aeruginosa and the genus Enterococcus, in particular Enterococcus faecalis, and from the yeast species Candida albicans.

5. The use according to claim 1, wherein the extracellular membrane vesicles are produced by a method comprising the following steps:
(a) culturing at least one probiotic under conditions suitable for the production of extracellular membrane vesicles,
(b) separating the at least one probiotic and extracellular membrane vesicles produced in step (a) and,
(c) purification and concentration of the extracellular membrane vesicles.

6. The use according to claim 5, wherein said purification and concentration step comprises at least one step selected from differential centrifugation, density gradient, exclusion chromatography, ultrafiltration, immunocapture and precipitation.

7. The use according to claim 1, wherein said material is selected from metals, metal alloys, polymers, glass, ceramic, and food.

8. A method for treating a surface of a material for preventing or reducing the formation of a biofilm on said surface, said method comprising a step of contacting extracellular membrane vesicles from at least one probiotic with said surface.

9. A material comprising at its surface extracellular membrane vesicles of at least one probiotic, or wherein extracellular membrane vesicles of at least one probiotic are incorporated.

10. The material according to claim 9, wherein said vesicles form a layer with a thickness of between 10 and 500 nm on said surface.

11. The material according to claim 9, selected from a packaging material, in particular food packaging, a water pipe, a heat exchanger, a pipeline, a catheter, a dressing, a cream and a medical device.
